(19) United States
(12) Patent Application Publication
(10) Pub. No.: US 2014/0099679 A1
(43) Pub. Date: Apr. 10, 2014

(54) SYSTEM AND METHOD FOR PRODUCING BIOMATERIALS

(71) Applicant: Renewable Process Technologies LLC, Devens, MA (US)

(72) Inventor: James Vincent Banks, Carlisle, MA (US)

(73) Assignee: Renewable Process Technologies LLC, Devens, MA (US)

(21) Appl. No.: 14/103,888

(22) Filed: Dec. 12, 2013

Related U.S. Application Data
(62) Division of application No. 13/022,152, filed on Feb. 7, 2011.
(60) Provisional application No. 61/302,402, filed on Feb. 8, 2010.

(51) Int. Cl. C12P 19/04; C12P 7/06; C12P 7/16; C12N 9/00

(52) U.S. Cl. CPC C12P 19/04; C12N 9/00; C12P 7/065; C12P 7/16
USPC 435/101; 435/183; 435/161; 435/160

(57) ABSTRACT

A bioreactor system for manufacturing and extracting a desired biomaterial from a microorganism by fermenting the microorganism in the bioreactor. The system includes a horizontal reactor vessel, one or more vertical discs rotatably mounted around a hollow shaft, a motor to power the shaft, and one or more spray nozzles arranged to spray required liquids on to the discs. The system is arranged so that the microorganism is not kept submerged within the reactor vessel during the fermentation process. The system is suitable for any type of microorganism, including fungi and bacteria, and can be modified to produce many types of desired biomaterials, including antibiotics, enzymes, ethanol, butanol, chitin, and chitosan. The method of the present invention generally provides steps for placing substrate on the vertical discs of the reactor vessel, inoculating the discs, introducing media, fermenting the microorganism, and extracting the desired biomaterial from the reactor vessel.

### CROSS-REFERENCE TO RELATED APPLICATION

The present invention claims priority to U.S. Provisional Patent Application Ser. No. 61/302,402, entitled “SYSTEM AND METHOD FOR PRODUCING BIOMATERIALS” filed Feb. 8, 2010. The contents of the related application are incorporated herein by reference.

### BACKGROUND OF THE INVENTION

1. Field of the Invention

The present invention relates to systems and methods for cultivating and extracting biomaterials from feedstocks. More particularly, the present invention relates to systems and methods for cultivating and extracting biomaterials from cellulistic feedstocks. The present invention is a system and related method for cultivating organisms in an un-submerged state and extracting biomaterials from the organisms.

2. Description of the Prior Art

Bioreactors are used to culture prokaryotic or eukaryotic cells to produce commercially important biomaterials through fermentation. Bioreactors can be employed in varying scales, up to and including at an industrial level. Given the complexities associated with mass-culturing living cells, optimal bioreactor design requires sophisticated engineering and intricate manipulations. For example, the bioreactor's environmental conditions such as gas (i.e., air, nitrogen, carbon dioxide, oxygen, or lack thereof) flow rates, nutrient levels, trace elements, temperature, pH and dissolved oxygen levels, must be evenly distributed throughout the reactor as well as closely monitored and controlled. Until now, large scale conventional stirred tank and solid state reactor designs required addressing competing engineering design issues relating to agitation speed/circulation rates, potential cell lysis under high shear conditions, high capital and operating costs, as well as heat and mass transfer issues with respect to higher viscosity liquid media and/or more complex solids matrices.

The type of bioreactor employed depends on the cell to be used, and the major types of bioreactors used in industry are stirred tank reactors (as mentioned above), bubble column reactors, air lift reactors, fluidized bed reactors, packed bed reactors, and flocculated cell reactors. These types of bioreactors are all submerged fermentation reactors, i.e., they all contain the fermentation substrate in liquid form, which is optimal for many bacterial and mammalian cells. However, none of these reactors are optimal for cultivating fungi or other organisms which may be better suited to be cultivated in their respective non-submerged states.

Unlike other eukaryotic organisms, fungi are composed of filaments called hyphae; their cells are long and thread-like and connected end-to-end. Another unique feature of fungi is the presence of chitin in their cell walls. Most fungi do not form spores in submerged fermentation bioreactors, and are therefore difficult to cultivate in such bioreactors. As such, fungi are usually cultured in solid-substrate fermentation bioreactors where the fungi are grown on organic materials. However, solid-state fermentation bioreactors are generally much more difficult to use than submerged fermentation bioreactors. Specifically, there can be problems with contamination, and control of the environment is difficult to achieve, particularly in relation to maintaining optimal and uniform temperature, gas, nutrient, moisture, product and by-product levels. While this application discusses the present invention in the context of fungi, it is to be understood that it is as applicable to other organisms of interest as noted herein.

An improved system for cultivating fungi and other organisms (including, but not limited to plants, animal cells, or bacteria) in order to produce biomaterials in an efficient manner is essential because of the commercial importance of many of the materials these organisms can produce. For example, fungi have long been used to produce antibiotics and various enzymes for industrial use or use in detergents. Furthermore, the vast commercial potential for chitin and chitosan (deacetylated chitin) derived from fungal cell walls is currently being realized. Commercial uses for chitin and chitosan include pharmaceuticals, wound care products, medical implants, agriculture, cosmetics, food additives, paper making, and textiles. Other examples of organisms that may be cultivated primarily in a non-submerged state are bacteria that metabolize cellulose and/or hemicellulose to produce ethanol or other commercial products.

As such, it is clear there exists a continuing need in the art for an improved bioreactor system that supports either fungi or microbes in a non-submerged state and method of harvesting biomaterials produced from the bioreactor system. Such a system will be robust from a microbial integrity perspective, improve upon current methods to load/unload substrate, as well as have greater capability to uniformly distribute high rates of both heat and mass transfer at selectable conditions determinable by the organism used and the output product desired. Many existing technologies address microbial integrity and aseptic processing adequately but may be labor intensive or inefficient to load/unload reactor substrate and product. Essentially all solid state reactors surveyed have a limitation of evenly distributing heat and mass transfer, which limits a given reactor's productivity and ability to uniformly produce high value product within specified operating ranges. The improved art of this invention addresses all significant solids (substrate) handling issues as well as heat and mass transfer issues. The net result is to increase the ease and yield of production of biomaterials in relation to current non-submerged bioreactor systems. Specifically, this invention permits production of particular products (examples include cellulase and hemicellulase enzymes, but the invention is not limited thereto) during the vegetative fungi growth phase. During this phase, water soluble products such as sugars and enzymes can be easily separated from the cell mass and substrate. Depending upon the needs of the organism, during this growth phase, all or a fraction of the water soluble products may be re-introduced back into the reactor. This would typically be accomplished by adding these products into the liquid media. Following the vegetative phase, the production of chitin can be optimized by initiating and supporting a fruit bearing phase. Once the fruit bearing phase is complete, the reactor can be operated to remove any remaining sugars and enzymes and efficiently harvest and remove the cell mass from the reactor vessel. An example of such a system will dramatically increase the amount of chitin and chitosan available in comparison to the current commercial source, shellfish waste, as well as avoid the problems associated therewith, such as seasonal supply, high processing costs, and complications in pharmaceutical or food use associated with shellfish allergies.

### SUMMARY OF THE INVENTION

It is an object of the present invention to provide an improved bioreactor system used to cultivate fungi, microbes, plants, animal cells, or bacteria primarily in a non-submerged state. It is another object of the invention to enable the user of the system to manufacture and extract biomaterials from cellulistic feedstocks when the cellulose itself is the feedstock and/or the substrate, or an inert material such as nylon fibers, or fibers made from other hydrophilic material, is the substrate and nutrients are applied accordingly. The present invention is suitable for use with other feedstocks as well. These and other objects are achieved with the present invention, which enables the user to cultivate large amounts of desired biomaterials in a simplified manner with the bioreactor of the present invention. The improved bioreactor is suitable for cultivating any biomaterials derivable from fungi including, but not limited to, antibiotics, enzymes, chitin, and chitosan.

The present invention is a system comprising a bioreactor for fungi and/or other microorganisms (including, but not limited to plants, animal cells, or bacteria) capable of thriving in a non-submerged state, and a method of using the same to manufacture and extract biomaterials from cellulistic feedstocks. More particularly, the present invention provides expanded surface area needed for high capacity output through a system comprising a horizontal reactor vessel with vertical discs layered with substrate upon both sides of the discs. Heat and mass transfer through the substrate (which is needed to sustain growth) is enhanced through the use of a hollow shaft that can support passage of both liquid and gasses through the substrate in either direction. The substrate is supported by the discs that are mounted upon the hollow shaft which in turn rotates to afford the substrate to be efficiently drained of excess moisture.

The arrangement of the system of the present invention also enables more uniform heat and mass transfer than what is capable from other solid state reactor geometries and configurations. In one embodiment, the system typically includes two integrated process modules; a reactor module and a support module. The reactor module includes a substantially horizontally arranged reactor vessel with substantially vertically oriented discs, which may also be referred to as trays, mounted on a substantially centered rotatable shaft. The reactor vessel also includes a well located in the lower tangent of the reactor vessel, and one or more manifolds, each manifold including one or more spray nozzles oriented along the axis of the vessel to distribute nutrient media and other fluids to the individual discs at appropriate times. The well facilitates efficient gas/liquid separation whereby excess liquid can be removed through the well while the bulk gas flow continues to pass through the vertical discs, substrate, and cell mass. The manifolds may be run within the interior or on the exterior of the reactor vessel. The reactor vessel further includes process piping suitable to permit communication between the reactor and support module while remaining isolated from the surrounding environment. The support module includes one or more receiver (media surge) vessels, one or more pumps for enabling the transport of liquids, one or more blowers for the transport of gases, one or more heat exchangers to enable the heating and cooling of liquids and gases in isolation from the process, and one or more membranes to enable the separation of cells and macromolecules from media and to provide a semi-permeable barrier that isolates the process from the outside environment.

The rotatable shaft of the reactor vessel may be rotated by a motor. The hollow shaft runs substantially or completely through the length of the reaction vessel. In communication with the rotatable shaft within the reactor vessel is a plurality of the vertically oriented discs, which may be single-sided but are preferably two-sided to maximize output product production. In relation to the vertical discs are the one or more manifolds, which may also be substantially horizontally aligned so as to be substantially parallel with the rotatable hollow shaft. The one or more manifolds include the spray nozzles in either the upper or upper and lower regions of the reactor vessel. As indicated, the system also includes a media surge tank, a pump, a membrane, centrifugation, or other means of cell separation and/or a means of molecular concentration, a tempering exchanger to either heat or cool the system, a gas phase heat exchanger, gas filters, blower, a means for humidity control, and a means to provide media, substrate and inoculum to the bioreactor system.

The reactor is a solid state reactor. The discs are preferably made of a semi-permeable material upon which a substrate, such as a cellulistic feedstock substrate, such as cellulose fibers, can be overlaid. Other materials determined to be suitable for the growth of organisms of interest may also be employed to establish the substrate of the vertically oriented discs. Fungi or other suitable microbes of interest are cultivated within the substrate matrix. The temperature and amount of spray delivered to each disc surface can be precisely controlled and monitored by application of suitable instrumentation, control hardware and algorithms. One or more fluids of interest are delivered to the interior of the reactor vessel by way of one or more external conduits in fluid communication with the one or more manifolds including the spray nozzles. The spray nozzles are preferably configured to provide substantially even distribution of the fluid(s) of interest over all discs. That substantially even distribution is further enabled by rotating the discs using the rotating hollow shaft. Enhanced heat and mass transfer is achieved by gas and liquid passing through the substrate into and through the interiors of the disc to the hollow shaft, which has a plurality of pores and which thereby provides an avenue of transport of such gas and liquid out of the reactor vessel. In one embodiment of the invention, a baffle is located within the hollow shaft and extends along its axis. The baffle divides the shaft in two or more zones that enable, for example, differing operating pressures across the semi-permeable membranes of one or more discs or sets of discs based upon rotation about the shaft.

The media surge tank is an agitated vessel equipped with a means of heat transfer that enables the media surge tank content to be heated or cooled on demand. One suitable means of heat transfer is a heat transfer jacket, although any suitable means of heat transfer known in the art may be used. Typically, this vessel is used to add media with fresh nutrients, substrate slurry, and any other additive that is to be pumped into the reactor. For example, the media may simply be nutritional media, or the media may contain additional materials, such as cellulistic fibrous material for example, so as to add substrate to the discs. Depending upon the step within the reactor operational cycle, the pump transfers materials from the media surge tank directly into the reactor or into the reactor via the spray nozzles of the one or more manifolds. After the desired operation has occurred with the reactor vessel, the pump, or some other device suitable for transferring a fluid from one location to another, may be activated to deliver liquid to the cell separator/molecular concentration operation for collection of the desired biomaterial. In one embodiment of the invention, a suspension of liquid and substrate is added to the reactor to the extent that the reactor is filled or partially filled with the suspension. The volume of the reactor is then reduced by drawing liquid through the semi permeable support material on discs and through the hollow shaft thereby permitting the previously suspended substrate to be deposited at the discs. The solids concentration, rate of deposition, and size distribution of the suspended substrate can be manipulated to tailor the depth and porosity of the applied substrate for the desired application. An important attribute this reactor system has over existing stirred tank reactor and other solid state reactor designs is that the reactor liquid media, which contains dissolved products of sugar(s) and enzymes, can be easily separated from the cell mass. The liquid media can then be processed with separation techniques known to those of skill in the art to remove enzyme and sugar(s) products as required during a fermentation process.

The cell separation/molecular concentration step is normally a two-step operation. The first step typically utilizes either centrifugation or a membrane-based microporous device to concentrate particles and thereby remove particulate from the clarified liquid fraction. The second step typically utilizes one or more membrane-based ultrafiltration and/or ion exchange chromatography steps and related devices to concentrate and diafilter macromolecules from the clarified product of the first step. In ultrafiltration practice, the solution containing enzymes, water, sugar, and salts, all of which can be fed into the ultrafilter membrane. Low molecular weight material (sugars, water, and salts) will pass through the membrane as a clarified liquid fraction. Larger molecular weight products, such as enzymes, will be concentrated as they are retained along with a fraction of the feed. Enzymes may be returned to the reactor vessel or removed for further processing as desired.

The trim heat exchanger is typically used to cool the fermentation media as it is re-circulated from the media surge tank, either through the ultrafilter, or bypassing the ultrafilter and prior to being sprayed onto the fungi and substrate. This heat exchanger, or dedicated heat exchangers, for each task may be the primary means of heating and cooling the reactor vessel during the reaction process.

The gas phase heat exchanger is designed to cool the gases prior to entry or re-entry into the reactor vessel to a temperature at or near its dew point. The closer the gas is to its dew point the less drying effect the gas has as it passes through the fermentation media and substrate.

The gas filters are used to provide a sterile barrier between the fermentation media and the surrounding environment. For example, one gas filter may be used to provide a sterile barrier between the fermentation media and the source for gas (air, oxygen, carbon dioxide, or a combination or sequence of two or more constituent gases) addition. The presence of gas filters aids in preventing contamination of the fermentation with external microbial factors. It is also useful in preventing contamination of the external environment with the cultured organisms (such as genetically modified organisms). In one embodiment of the invention, the gas of interest is applied into the liquid prior to its application to the discs. This method may be accomplished by sparging gas into the surge tank or by adding gas directly into the liquid stream. The motive force for this embodiment may be achieved using a gas blower or other suitable means of delivering pressurized gas into the process.

The blower is configured to generate a driving force for gas passing through the reactor vessel and through the substrate and fermentation media. In one embodiment, the blower may be designed to operate with a variable frequency drive as well as operate in both forward and reverse directions. In another embodiment, two or more blowers may be added to achieve the objective of forward and reverse operation. The utilization of the blower aids in the efficiency of the method of the present invention. When operating near saturated air conditions, a blower operating with mostly recirculated gas stream requires less energy than a bone dry gas that must be continuously humidified. Further when compared with conventional stirred tank reactor of comparable capacity, a solid state reactor using a blower such as exists with the system of the present invention, requires only a fraction of the energy required when sparging a conventional stirred tank reactor capable of equivalent biomaterial output. In one embodiment of the invention, relative humidity of the gas delivered to the reactor vessel is controlled to the desired level. This may be accomplished by adding low pressure steam to increase the amount of absolute moisture into the gas stream and/or by cooling the gas stream to increase its relative humidity.

The present invention provides a means to apply a substrate, sterilize the hardware and substrate, inoculate the substrate, and a heat and mass transfer capability to support the metabolic needs of the inoculated microbe. It further enables periodic harvesting of all or some of the product(s) expressed or generated by the microbes, and it provides a means of removing substantially all the product and substrate followed by downstream concentration and purification. A means is also optionally provided to fully clean the process equipment following the final harvest of product from the reactor system. Electronic process control hardware and software may be employed and integrated with the system to allow persons knowledgeable in the art to make incremental additions and/or deletion to the following sequence of process steps, wherein specific operating conditions may be designated to produce biomaterials interest.

The following steps are carried out in an embodiment of the method of the present invention. First, the cellulose-based or other substrate of interest, including an inert substrate, is added to the screens of the vertical discs in the reactor vessel and sterilized. It is to be noted that more than one substrate may be employed if that is determined to be suitable for the biomaterial to be produced. Next, the substrate is inoculated with the desired fungi or other microorganism. Media is then introduced into the reactor system and sprayed on the substrate, followed by the reaction step. If the reaction is a fungal fermentation, the reaction typically occurs in three phases, the incubation phase, the vegetative growth phase (also known as the enzyme expression phase) and the fruit bearing phase. The incubation phase typically lasts three days following inoculation. In a typical incubation phase, thread-like hyphae form throughout the substrate. In the vegetative growth phase, the hyphae mature into mycelium. This vegetative phase typically lasts 15 to 20 days and is the period of greatest enzyme and sugar production. The final phase is the fruit bearing phase, where media can be altered to promote production of cell mass containing fruit. In each phase, the media introduced into the reactor vessel is conditioned as desired with sugar content, trace nutrients, temperature, pH, pressure, flow rate and gas concentration parameters carefully controlled and monitored. In the vegetative phase, the same parameters are controlled and, in addition, sugar is removed from the media through the cell separation/molecular concentration steps. It is important to note that some sugar is needed to support the metabolism of the fungi, but too much sugar will retard the enzyme expression rate. The final step of the method is to carry out post-fermentation operations, which may include recovery of the biomaterial (such as enzyme and/or chitin), cleaning in place, or inactivation. In one embodiment of the invention, the reactor is inoculated with bacteria in lieu of fungi. Depending upon the microorganism and the metabolic pathway, cellulase enzymes may be added to the reactor to provide a means of converting cellulose to glucose, or the microbe may be capable of metabolizing cellulose to produce the desired biomaterial directly. This conversion process may be completed in either an aerobic or an anaerobic embodiment of the present reactor. Further, the present reactor may be operated to produce either ethanol or butanol biomaterial.

The configuration of the system of the present invention also provides advantages in the induction or enzyme expression phase when using the system. Applying light at a specific frequency/wavelength and intensity to induce the biologically active organism growing in the bioreactor to produce the biomaterial of interest can be advantageous in increasing the speed of production or altering the metabolism of the organism. The system of the present invention has superior geometry attributes over other solid state reactors or liquid fermentation systems in relation to the application of light. Specifically, in some embodiments, the system of the present invention may be configured to include sight glasses installed along the walls of the reactor vessel. Lamps or lights comprising light emitting diodes (LEDs) or any suitable light source known in the art can be configured to illuminate the interior of the reactor. The configuration of the system of the present invention provides that light will be transmitted efficiently and evenly, and therefore production scale operations can be carried out in a more economical manner to the extent that the introduction of light improves the biological activity as desired. Lamps or lights may be acquired or engineered to radiate light at a specific wavelength and intensity depending on the metabolic requirements of the process. This embodiment of the invention is suitable for use with a solid state reactor operating with any type of microorganism, including bacterial, plant, or fungi organisms.

The present invention is directed to a system and related method for the un-submerged production of biomaterials of interest in an efficient and effective manner. The invention will be more fully understood upon review of the following detailed description, the accompanying drawings, and the appended claims.

### BRIEF DESCRIPTION OF THE DRAWINGS

FIG. 1 schematically illustrates a system for manufacturing and extracting biomaterials according to one embodiment of the invention. Note that this illustration provides a spray nozzle manifold mounted internally within the reactor vessel. An alternate embodiment to the invention provides the means to supply the nozzles through a manifold external to the vessel.

FIG. 2 is a cross sectional interior view of the reactor vessel according to one embodiment of the invention, showing the discs and the action of the spray nozzles. An alternate embodiment to the invention locates the nozzles above the discs or at some intermediate point, and spraying down upon the discs. Note that in either embodiment, spray nozzles are configured to spray both sides of each disc.

FIG. 3 is a side view of the reactor vessel, discs and hollow shaft of the invention, showing the entrance and exit points of liquids and solids. An alternate embodiment to the invention provides the means of adding substrate, as a high suspended solids slurry, to the reactor vessel through one or more ports that bypass the spray nozzles.

FIG. 4 is a simplified flow diagram showing primary steps of one embodiment of the method of the invention for manufacturing and extracting a desired biomaterial.

### DETAILED DESCRIPTION OF THE PREFERRED EMBODIMENTS OF THE INVENTION

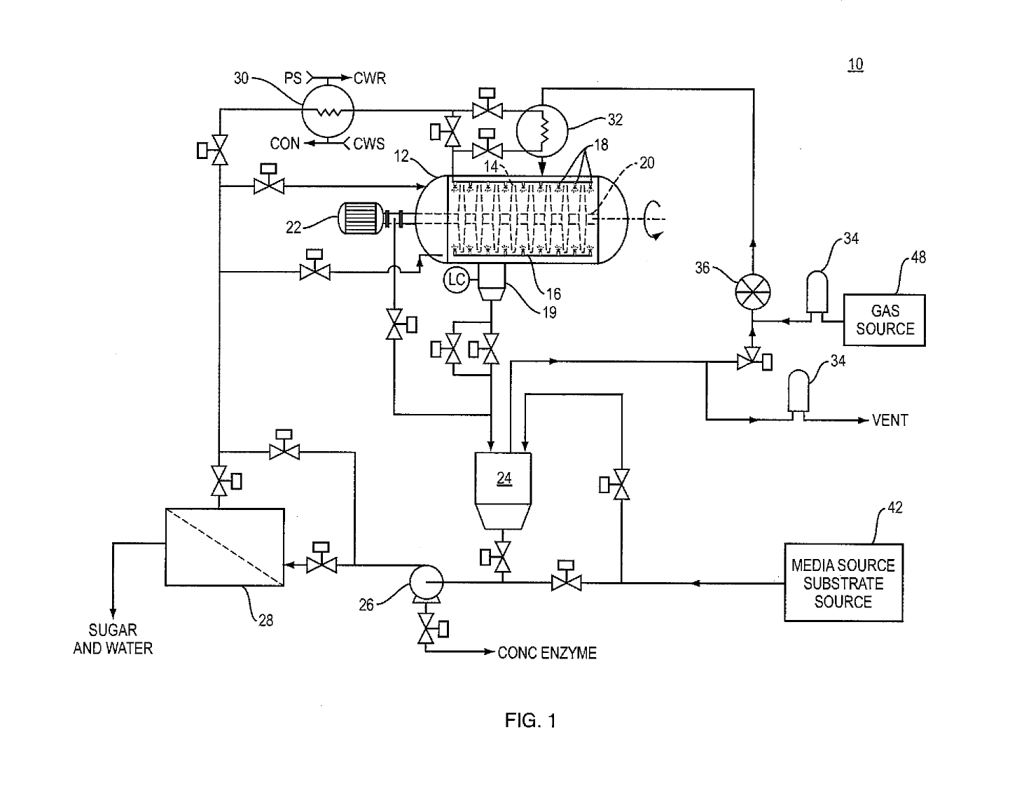

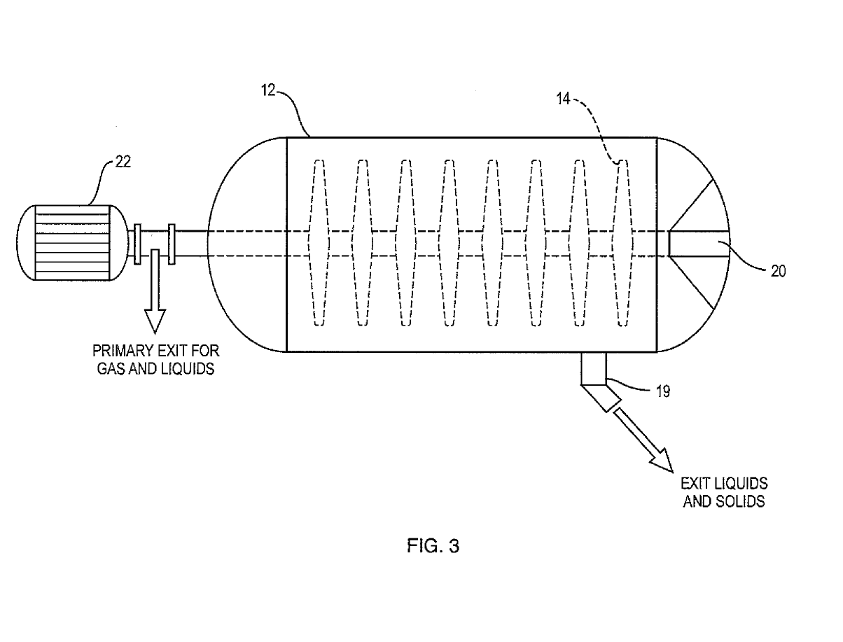

A system 10 of the present invention suitable for manufacturing and extracting desired biomaterials from one or more microorganisms including, but not limited to, fungi, is shown in FIG. 1. The system 10 includes a horizontal reactor vessel 12 containing vertical trays or discs 14 and a horizontal manifold 16 containing spray nozzles 18 in either the upper or upper and lower regions of the reactor vessel 12. The reactor vessel 12 also includes a well 19 located in the reactor vessel 12. The well 19, which is also shown in FIG. 3, is equipped with means to control the fluid level in the well 19 and/or the reactor vessel 12. Suitable means to control the fluid level are instruments and flow control devices designed to sense and/or maintain the liquid level in the well 19 while preventing the liquid volume from becoming too high or too low within the well 19 or vessel 12 at any given step in the process.

Suitable instruments and flow control devices for use in the well 19 as means to control the fluid level are known in the art and include, for example a differential level transmitter, level control valves, and a computerized level controller. A differential level transmitter is a level sensing device that accurately measures the weight of a column of liquid between an upper and a lower sensor. The upper sensor, which may be one or more upper sensors, is located in an upper region of the vessel 12 and is used to monitor system pressure. The lower sensor, which may be one or more lower sensors, measures the pressure in a lower region of the well 19 and/or vessel 12. Pressure at the lower sensor equals the system pressure plus the weight of any liquid residing above the lower sensor. Therefore, fluid level is proportional to the differential pressure between the upper and lower sensors. In this application, level control valves may include a pair of valves with each valve capable of modulating 0 to 100% open and thereby handling the regulation of the full range of high and low flow requirements. The level sensor and control valve(s) are interfaced with the computerized level controller that is capable of modulating the control valve output based upon the sensor input and a desired fluid level set point. As the fluid level rises above the desired set point, the level controller signals the level valve(s) to modulate open. Conversely, if the fluid level falls below the level set point, the controller will signal the level valve(s) to modulate closed.

The system 10 also includes a hollow shaft 20 running through the reactor vessel 12 and operable by a motor 22. The system 10 further includes a media surge tank 24, a pump 26, a membrane system 28, a trim heat exchanger 30, one or more gas phase heat exchangers 32, one or more gas filters 34 and a blower 36. The combination of these components of the system 10 arranged and configured as described herein enable a user to produce desired biomaterials from microorganisms in a non-submerged manner not enabled by existing bioreactors.

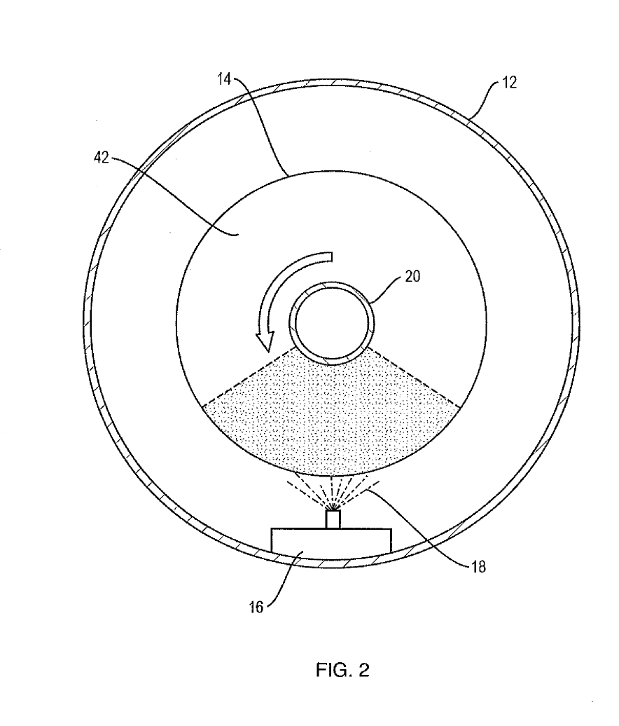

As further shown in FIGS. 2 and 3, the discs 14 of the reactor vessel 12 are mounted rotatably on the hollow shaft 20 so they can be variably reached by the spray nozzles 18 mounted on the horizontal manifold 16 in the reactor vessel 12. Note that in the embodiments of the invention, the spray nozzles 18 are configured to spray both sides of each disc 14. Optionally, each disc 14 may only be sprayed on one side. The driving force for supply pressure and flow to the spray nozzles 18 is regulated based upon the process requirements of the sequence. For example, during the reaction step, which may be referred to herein as the fermentation step, pressure drop across the spray nozzles 18 is typically at its lowest. In the final harvest step where substrate 42 that had been applied to the disc 14 is mechanically removed from a screen support layer of the disc 14, the nozzle pressure is typically at a significantly higher pressure. The means of pressure control to the nozzles 18 is typically achieved through regulating the motor speed of pump 26. However in an alternate embodiment, pump 26 may be operated at a fixed speed and a pressure control valve, located between the pump 26 and manifold 16, can be applied to regulate nozzle flow and pressure. The number and configuration of the spray nozzles is determined by the size of the reactor vessel 12, the particular output function of the system 10 and such other parameters as considered of importance for a specific process.

The preferred substantially vertical orientation of the rotating discs 14 permits the discs 14 to be efficiently sprayed from the location of the spray nozzles 18. Furthermore, the configuration of the reactor vessel 12 in the system 10 allows for complete saturation of the discs 14 without any adverse effects from “over spray” or excess fluid added to the vessel 12. For example, in comparison to a horizontal disc or tray configuration, the vertical disc 14 configuration prevents any pooling of fluids on the substrate 42, which can have harmful effects on the microorganism. The sprayer nozzles 18 are used to saturate the vertical discs 14 with liquid including nutrient media. The liquid may include additional substrate, such as in the form of cellulistic fibers, in order to add substrate 42 to the vertical discs 14. An additional benefit conferred by the use of spray nozzles 18 to apply media to the microorganism is that the liquid spray helps cool the biomass under reaction, aiding in optimizing production of the desired biomaterial.

Other benefits of the configuration of the system 10 of the present invention are conferred by the use of the hollow shaft 20 through which the flow of gas and liquid can be controlled. For example, it may be advantageous to permit gas or liquid to flow through the shaft 20 at various points along its rotation to remove excess liquid or, in another embodiment of the invention, collect liquid sprayed upon the discs 14 used to collect an expressed enzyme of interest. This liquid maybe pure nutrient or it may contain a surfactant to enhance the release of enzyme from the biomass and substrate 42. The rate of liquid flow can be controlled by manifolding sections of the shaft 20 through the use of an internal baffle mechanism within the hollow shaft 20, or by adjusting the driving force by manipulating blower speed and the relative pressures of reactor and surge tank vessels 12 and 24. A further embodiment of the invention includes the option of using the reverse flow of gas from inside the disc 14 (supplied through the hollow shaft 20) through the substrate 42 and biomass. This technique, along with the vertical orientation of the discs 14, and high pressure spray nozzles 18 assists in removing both the biomass and substrate 42 from the support structure comprising each side of the discs 14.

As noted, the amount of gas and liquid flowing through the vertical discs 14 can be controlled as the discs 14 rotate around the hollow shaft 20. As shown in FIG. 2 for example, the spray predominantly contacts the bottom third of the discs 14 (illustrated by shading) as the discs 14 rotate counterclockwise around the hollow shaft 20. Localized spray and bulk gas flow through the shaft 20 are beneficial for biomaterial growth in that they create significant gas mixing within the reactor vessel 12 in comparison to stationary horizontal tray reactors.

In one embodiment of the invention, baffles may be employed with the reactor vessel 12 in order to induce greater mixing and transfer of gases through the disc surface. The baffles are preferably are located at either end of the reactor vessel 12, which may be of a domed configuration, to prevent air flowing from the top to the bottom of the reactor vessel 12 to substantially bypass the discs 14 by traveling along the vessel ends that are unoccupied by discs 14. Enhanced gas flow can also be achieved as fermentation progresses and the biomass and substrate 42 become more mechanically bound in a stable matrix by increasing the rotational speed of the discs 14 as well as the frequency and magnitude of the gas flow. Gas mixing can be further enhanced within the reactor vessel 12 by using a bidirectional gas flow, such as from the outside to inside of the disc 14 and alternately from the inside of the disc 14 to the outside of the disc 14, as well as from the top of the reactor vessel 12 to the bottom of the reactor vessel 12.

The source for bulk air flow is two-fold. First, gas blower 36 provides the bulk gas supply for the enhanced gas flow. Typically, this gas flow is achieved through recirculation of the gas from the receiver (media surge) vessel 24 to the reactor vessel 12. A fraction of the total gas flow from the reactor vessel 12 will be purged to a vent and an equal amount will made up from a fresh gas source comprised typically from one or more of the following process gas streams: air, nitrogen, carbon dioxide, or oxygen. Gas flow pressure and rates can vary based upon the process (metabolic) requirements. The direction of bulk gas flow is controlled by one of three means: either by the direction (rotation) a reversible blower operates (clockwise vs. counterclockwise rotation) or through the configuration of a valve nest around a unidirectional blower, or by employing two blowers, one for each forward and reverse directions.

In one embodiment of the invention, sight glasses may be incorporated into the system 10 to alter the metabolism of the organism by allowing light of a particular wavelength or frequency to shine on the discs 14. The reactor vessel 12 may include sight glasses or windows in the walls of the reactor vessel 12, and lamps or lights can be positioned to shine through these portions using methods and equipment known in the art. Alternatively, lamps or lights may be included internally in the reactor vessel 12 and controlled externally using methods and equipment known in the art.

The vertical disc 14, spray nozzle 18, and hollow shaft 20 configuration of the system 10 lends itself to easier loading of the substrate 42 and inoculum, as well as simplified solid/liquid separation during fermentation. Additionally, the configuration also lends itself to dislodging and removal of the substrate 42 and cell mass following fermentation. In one example, this configuration makes the system 10 well suited for producing enzymes during the vegetative growth phase and chitin from fungal cell mass following the fruit bearing phase of a fungal reactor cycle. In this configuration, vertical discs 14 support both substrate and mycelium and thereby permit heat and mass transfer into the substrate/mycelium matrix on the discs 14 as gas and liquid pass through the discs 14 and/or hollow shaft 20, and are delivered to media surge tank 24. The liquid media returned to the media surge tank 24 contain sugars and enzymes and may be returned to the reactor vessel 12 or removed using the membrane system 28 as desired. In some embodiments, the fermentation mass can be easily dislodged from the vertical discs 14 with a high pressure spray from the spray nozzles 18, and the overall design of the discs 14 in the reactor vessel 12 allows the user to exploit gravity to assist in delivering the solid bio-product to the bottom of the horizontal reactor vessel 12, where it can be sluiced from the reactor vessel 12 (as shown in FIG. 3). The semi-permeable material comprising the sides of the discs 14 mounted upon the hollow shaft 20 permits efficient heat and mass transfer through the substrate and cell mass as liquid and gaseous solutions pass from the vessel 12 interior through the discs 14 and exit the reactor vessel 12.

The discs 14 are preferably made of a semi-permeable material upon which the substrate 42, such as cellulose fibers from a cellulistic feedstock but not limited thereto, can be overlaid. The products of reaction are cultivated on the surface of the substrate 42. The discs are preferably two-sided but may be used in a single-sided arrangement, and the semi-permeable material may be a stainless steel wire screen capable of retaining the substrate 42 and the microorganism for growth on one or both sides. Other materials including metallic and nonmetallic materials may also be used to form the semi-permeable discs 14, provided there is some porosity to the material chosen. The discs 14 must also include a passageway or other form of communication means as a space between the two sides of the disc 14 to permit the flow of fluid (both liquid and gas) from the underside of the material through this passageway and into the hollow shaft 20. Similarly, if the discs 14 are formed as single structures made of two pieces of semi-permeable material joined at the outer edges thereof, it is necessary that there be included such a passageway between the two pieces. Other arrangements for the discs 14 may be employed provided they enable substrate and biomass support and allow fluid to pass through to the hollow shaft 20.

The media surge tank 24, powered by pump 26, is used to add media with fresh nutrients, substrate slurry, and any other desired fluid to the reactor vessel 12. The media surge tank 24 connects to the reactor vessel 12 via the trim heat exchanger 30 used to regulate the temperature of the incoming fluids. The media surge tank 24 is also used as a means to collect and separate liquid and gas fluids that flow out of the reactor vessel 12 through the hollow shaft 20. In the case of liquid, the surge tank 24 directs the liquid flowing from the hollow shaft 20 to pump 26 and a post-fermentation compartment of the system 10 such as the membrane system 28 or other suitable cell separation/molecular concentration means, where the desired biomaterial can be collected or the media filtered for re-use in the system 10. In the case of gas, the surge tank 24 directs the gas flowing from the hollow shaft 20 to the appropriate gas venting and fresh gas makeup and on to blower 36 where the gas is returned back to the reactor vessel 12. In one embodiment of the invention, a suitable means of controlling the relative humidity of the gas stream delivered to the reactor vessel 12 is provided. Relative humidity control includes the means of adding moisture through a low pressure steam addition as well as removing moisture by cooling and subsequent heating of the gas stream.

Membrane system 28 is defined as one or more membrane stages containing suitable microporous or ultrafilter elements or other appropriate collection means that are known in the art, and can be selected based on the particle size and molecular weight of biomaterial desired to be retained, such as particles or macromolecules (enzymes). A typical configuration for a membrane system 28 includes a 0.2 micron microfiltration first stage element(s) whereby the feed is delivered from media surge tank 24 and concentrated retentate is typically returned to the reactor system 10. Clarified media (permeate) is delivered as feed to the second stage ultrafiltration operation. A typical second stage configuration for a membrane system 28 includes a 20,000 molecular weight cutoff (MWCO) ultrafiltration second stage whereby the feed is first stage permeate and second stage concentrated retentate is rich in expressed enzymes with either a fraction or all typically returned to the reactor system 10. Ultrafiltration permeate typically contains water, salts and sugar, with either a fraction or all of this stream removed from the reactor system 10. In other embodiments of the invention, the membrane system 28 may include one or more of a centrifuge, chromatography operation, or some other separation technique known to those skilled in the art for use to remove particles and/or concentrate sugars and/or macromolecules.

When media is returned to the system 10 after filtration in the membrane system 28 it can be supplemented with additional nutrients or substrate, i.e., feedstock such as cellulistic feedstock. The configuration of the system 10 allows for re-bedding of the substrate 42 by adding sterilized substrate 42 to the media surge tank 24 and flooding the reactor vessel 12 with the liquid/solid slurry. As the permeate passes through the vertical discs 14, additional solids will be deposited on the substrate 42, and will be inoculated by the existing liquid contained in the reactor vessel 12. By carrying out this re-bedding process, the system 10 of the present invention can be operated for extended periods without the need of downtime for cleaning, media preparation, and the like.

If the media passing through the hollow shaft 20 is processed by the membrane system 28, one or more process streams may be returned to the reactor vessel 12 via the media surge tank 24 or via the trim heat exchanger 30. The trim heat exchanger 30 is typically used to regulate the fermentation media temperature as it is re-circulated from the media surge tank 24 or through the membrane system 28. In either case, the desired reactor temperature, through either heating or cooling, is achieved by spraying media onto the biomass and the substrate 42 on the vertical discs 14. The trim heat exchanger 30 is the primary means of heating and cooling the reactor vessel 12 during fermentation.

Whereas the trim heat exchanger 30 is used primarily to manipulate the temperature of liquids, the gas phase heat exchanger 32 is designed to heat or cool gases prior to entry or re-entry into the reactor vessel 12 to a temperature at or near its dew point. The gas from the gas source 48 for the gas phase heat exchanger 32 must first pass through a gas filter 34 in order to maintain a sterile barrier between the fermentation occurring in the reactor vessel 12 and the surrounding environment. Gases that are recirculated do not require similar sterile barrier filtration. The driving force for the gas entering and leaving the reactor vessel 12 is generated by the blower 36. In one embodiment of the invention, the blower 36 may be designed to operate with a variable frequency drive as well as operate in both forward and reverse directions. In another embodiment, two or more versions of the blower 36 may be added to achieve the objective of forward and reverse operation. In an embodiment of the invention, low pressure saturated steam or a heat transfer jacket maybe added to the media receiver (surge) vessel 24. In any of these embodiments, the temperature and vapor pressure of the gas contained in the head space of the media receiver (surge) vessel 24 can be controlled such that relative humidity of the gas supplied to the reactor vessel 12 is at or near saturation after passing through gas exchanger 32.

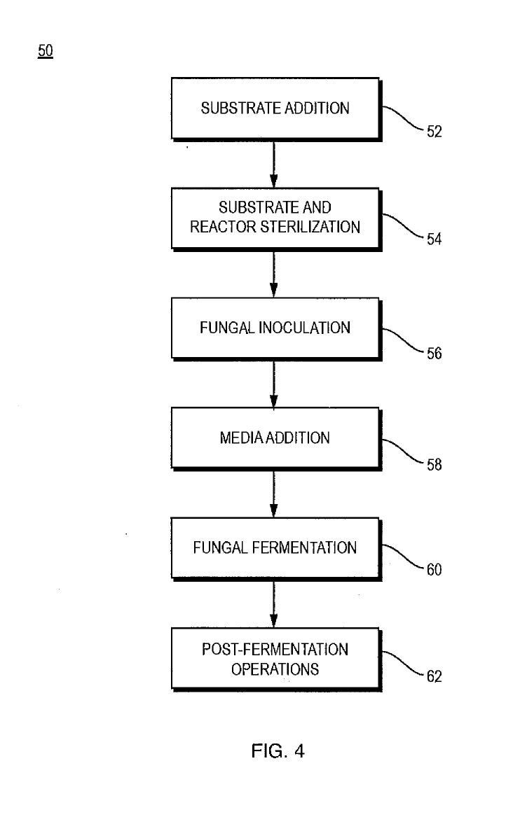

With reference to FIG. 4, in operation, the system 10 of the invention can be used in a method 50. In one embodiment the process steps of the method 50 may be automated. The method 50 of the invention includes the following steps, which are preferably to be carried out under aseptic conditions.

First, in step 52 the substrate 42 is added to the semi-permeable material (screens) of the vertical discs 14 in the reactor vessel 12. Substrate 42 can be any feedstock suitable for the desired biomaterial output. For example, the feedstock may be a cellulistic feedstock suitable for supporting fungal growth. In step 54, the substrate and reactor are sterilized in a manner, such as by using a sterilizing material such as a hydrogen peroxide (H₂O₂) solution, which is circulated through the reactor vessel 12. In another embodiment, pressurized saturated steam is applied in order to kill any microbes contained in or on the fiber of the substrate 42 and equipment inside the reactor vessel 12. After the sterilization, the reactor vessel 12 may be rinsed with a sterile buffer and drained. Next, in step 56, fungal or other microorganism inoculation is accomplished by adding the contents of a seed reactor to the reactor vessel 12. Media is then introduced into the reactor vessel 12 in step 58 through the media surge tank 24 as described above and sprayed on the substrate 42 by the spray nozzles 18. In another embodiment of this invention, pre-sterilized media containing cellulosic feedstock are inoculated and delivered to a pre-sterilized reactor system 10. The inoculated feedstock is delivered to the reactor vessel 12 and the inoculated feedstock 42 is deposited upon the semi-permeable material (screens) as liquid is removed from the hollow shaft 20.

The next step is microorganism fermentation, step 60. Fungal fermentation, for example, in step 60 occurs in three phases, the incubation phase, the vegetative growth phase and the fruit bearing phase. In a typical incubation phase, media are introduced into the reactor vessel 12 and are conditioned to stimulate formation of hyphae within the substrate 42. The sugar content, trace nutrients, temperature, pH, pressure, surge rate, and gas concentration parameters are carefully controlled. In the vegetative growth phase, hyphae mature into mycelium and enzyme expression is promoted, the same parameters are controlled, and in addition, sugar is removed from the media by draining the media through the hollow shaft 20 and removing excess sugar using membrane system 28. In this configuration, all or a portion of the enzymes delivered to the membrane system 28 may be returned to the reactor system 10. It is important to note that some sugar is needed to support the metabolism of the fungi, but too much sugar will retard the enzyme expression rate. In the fruit bearing phase, the cell mass is increased.

The final step of the method, step 62, is to carry out post-fermentation operations, which may include recovery of the biomaterial (such as enzyme and/or chitin), cleaning in place, or inactivation. A desired enzyme can be recovered by removing the liquid media from the reactor vessel 12 using the membrane system 28 as described above. This media has been circulated through the reactor vessel 12 by continuous spraying of the vertical discs 14 with the spray nozzles 18. Maximum enzyme recovery can be ensured by rinsing the substrate 42 and biomass with a buffer, or a buffer containing surfactant. After the desired enzymes have been removed, the desired biomaterials, such as chitin, can be recovered from the biomass, such as fungal material. A sodium hydroxide (NaOH) solution is introduced into the reactor vessel 12, and the biomass and substrate 42 are separated from the discs 14 by mechanical means or by high pressure spray with the NaOH solution. The material recovered from that step is removed from the reactor vessel 12 and processed using high shear mixers or other means known in the art to remove desired bio product from the biomass.

The present invention has been described with respect to various examples. Nevertheless, it will be understood that various modifications may be made without departing from the spirit and scope of the invention. Accordingly, other embodiments are within the scope of the claims appended hereto.

1-20. (canceled)

21. A method for manufacturing and extracting a desired biomaterial from a microorganism in a system comprising a horizontal reactor vessel, one or more vertical discs rotatably mounted around a hollow shaft within the reactor vessel, one or more spray nozzles mounted on a manifold within the reactor vessel, a motor to power the rotation of the shaft, a media surge tank, and a transfer device to supply liquid including the microorganism to the spray nozzles and/or to the reactor vessel, the method including the steps of:
adding substrate to the one or more vertical discs;
inoculating the system with the desired microorganism;
introducing media into the reactor vessel by spraying the discs with the liquid through the one or more spray nozzles;
cultivating the microorganism; and
recovering the desired biomaterial from the reactor vessel.

22. The method of claim 21, wherein the one or more vertical discs are made of a semi-permeable material.

23. The method of claim 21, wherein the step of adding substrate to the one or more vertical discs includes adding a substrate derived from a feedstock.

24. The method of claim 21, wherein the step of recovering the desired biomaterial includes using a membrane system.

25. The method of claim 21, wherein the step of recovering the desired biomaterial includes using high pressure spray to dislodge the microorganism and the substrate from the one or more vertical discs.

26. The method of claim 21, wherein the step of introducing media into the reactor vessel includes using a trim heat exchanger to modulate the temperature of the liquid supplied to the reactor vessel.

27. The method of claim 21, wherein the step of cultivating the microorganism includes using one or more gas phase heat exchangers, one or more gas filters, and one or more blowers.

28. The method of claim 21, wherein the step of cultivating the microorganism includes using methods of adding gas directly to the liquid prior applying the liquid to the discs.

29. The method of claim 21, wherein the microorganism is fungi and the desired biomaterial is chitin or chitosan.

30. The method of claim 29, wherein the microorganism is fungi and the desired biomaterial is an enzyme.

31. The method of claim 21, wherein the microorganism is bacteria and the desired biomaterial is ethanol or butanol.

32. The method of claim 21, further comprising the step of enabling the shining of light onto the one or more vertical discs.

33. The method of claim 21, wherein the step of inoculating the reactor vessel with the desired microorganism includes inoculating pre-sterilized substrate feedstock with the desired microorganism and then adding the substrate to the one or more vertical discs.

34. The method of claim 21, wherein the vertical discs are configured to support the substrate and the microorganism to enhance the separation of enzymes from the liquid.

35. The method of claim 21, wherein the vertical discs are configured to support the substrate and the microorganism to enhance the separation of sugars from the liquid.

36. The method of claim 21, wherein the vertical discs are configured to establish optimal metabolic conditions to enhance heat transfer to the microorganism.

37. The method of claim 21, wherein the vertical discs are configured to establish optimal metabolic conditions to enhance mass transfer to the microorganism.

38. The method of claim 21, further comprising the steps of passing one or more gasses and/or liquids from the reactor vessel through the substrate and microorganism and removing the one or more gasses and/or liquids from the reactor vessel through the hollow shaft.

39. The method of claim 21, wherein the step of recovering the desired biomaterial includes using gas and/or liquid flow reversal, where the gas and/or liquid flow from within the discs to the outside of the discs.

40. The method of claim 21, wherein the reactor vessel contains a well located along the lower tangent of the vessel and this well contains means to control the fluid level within the well and/or the reactor vessel.

41. The method of claim 21, wherein an inert hydrophilic substrate is applied to the vertical discs, liquid media is applied to the substrate, and the reactor is inoculated permitting the microorganism to attach themselves to the substrate.
